(19) United States
(12) Patent Application Publication
(10) Pub. No.: US 2016/0101064 A1
(43) Pub. Date: Apr. 14, 2016

(54) PROCESS FOR PREPARING AN EMULSION OF AN ACTIVE INGREDIENT AND PARTICLES OBTAINED FROM THIS EMULSION

(71) Applicant: ADISSEO FRANCE S.A.S., Antony (FR)

(72) Inventors: Jean-Marie DOLLAT, Montlucon (FR); Geraldine LAFITTE, Begues (FR); Emilie RUFFIN, Dijon (FR); Odile CHAMBIN, Dijon (FR)

(21) Appl. No.: 14/893,051

(22) PCT Filed: May 21, 2014
(86) PCT No.: PCT/FR2014/051190

§ 371 Date: Nov. 20, 2015

(30) Foreign Application Priority Data
May 21, 2013 (FR) 1354539

(51) Int. Cl. A61K 31/07; A61K 9/16; A61K 9/113

(52) U.S. Cl. CPC A61K 31/07; A61K 9/113; A61K 9/1694; A61K 9/1658; A61K 9/1652

(57) ABSTRACT

The invention concerns a method for preparing a double emulsion of a fat-soluble and hydrophobic active ingredient, which comprises the following steps:
(a) Providing an aqueous solution of at least one whey protein;
(b) Providing an aqueous solution of at least one anionic polysaccharide;
(c) Subjecting the whey protein(s) to a denaturation treatment;
(d) Hot-preparing an o/w emulsion of the active ingredient in the solution (c), then hot-preparing an (o/w)/w emulsion of the o/w emulsion in the solution (b), or
(e) Preparing an w/w emulsion of the solutions (c) and (b), then hot-preparing an o/(w/w) emulsion of the active ingredient in the w/w emulsion.

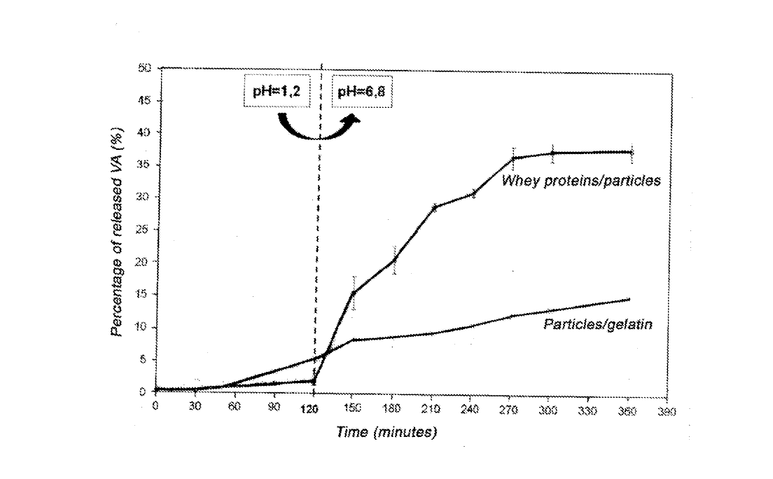

It also concerns the manufacturing of particles of said active ingredient from a thus prepared emulsion, and the obtained particles.

### TECHNICAL FIELD

The invention concerns particles of hydrophobic and fat-soluble active ingredients, and their manufacturing method.

### BACKGROUND

These molecules, such as vitamins, fatty acids, are very widely used in many technical fields such as pharmaceutical, cosmetic, agri-food industries, and in particular in the field of animal nutrition. For example, vitamins A and E are commonly used for preparing food promoting animal growth.

Their hydrophobic nature and their environmental fragility, in particular thermal and chemical, both during their formulation and their storage as well as during their use, make their encapsulation necessary.

The present invention is hereinafter more particularly described with reference to vitamin A, but of course, its frame is not restricted thereto, and it applies to any hydrophobic and fat-soluble active substance and any mixture of such substances.

The vitamin A exists in several forms, in particular in the state of ester, and and it is in one of its more stable forms, the retinyl acetate, that it is the most often consumed by farm animals (poultry, pigs and bovines). However, it remains sensitive to oxidation, to temperature, to light, to acids. In pharmaceutical application or in animal nutrition, it is thus very quickly degraded as soon as it comes into contact with the first severe conditions, in particular acids, of the digestive system, which does not make a bioavailable form of the vitamin A.

In order to preserve at best these sensitive active ingredients, it is known for a long time to protect them by coating or encapsulation. Various ways of encapsulating vitamin A have been developed and widely used, such as that implying proteins, and in particular gelatin: vitamin A is mixed with gelatin, then a gelatin cross-linking is caused allowing to obtain, by atomization or double emulsion, vitamin A particles.

One of the aims of the present invention is to obtain a protected form, or particle, of hydrophobic and fat-soluble active ingredients, without using gelatin, while preserving the benefit of the latter, in particular its protective properties, its easy supply and its simple handling. The use of gelatin requires that of cross-linking agent such as glutaraldehyde, and the present invention seeks to be free from such an agent. The invention further aims the implementation of an industrializable manufacture and environmentally friendly method, in order to obtain such a form of an active ingredient.

The substitution of gelatin by one or many biopolymer(s) has to respond to the following exigencies:
The particles have to comprise a vitamin content of at least 1,000,000 Ul/g;
The encapsulation yield should approach 100%;
The active ingredient has to be physico-chemically stable and bio-available.

According to the application filed of the active ingredient, in particular if it is intended for animal nutrition and thus to be incorporated in a premix, the obtained particles have to be of small size, preferably a size comprised between 50 and 1000 μm, advantageously less than 800 μm and even better in the order of 300 μm. They have furthermore to possess a low residual humidity, preferably less than 8% and to be water-insoluble.

The above requirements have been approached by methods involving milk proteins such as caseins and whey proteins, in order to replace gelatin. Thus, the document WO01/47560A2 describes a method for manufacturing particles of fat-soluble vitamin, such as vitamin A, consisting in obtaining an o/w emulsion by adding an oil solution of said vitamin in an aqueous suspension of a gelling agent which is preferably a carrageenan and of a protein, which may be a milk protein such as a casein or lactoglobulin; the particles are then formed by double emulsion (o/w/o) or atomization.

Increasingly more stable and more concentrated forms of fat-soluble and hydrophobic active ingredients are still sought, and this is an aim of the present invention.

The authors have developed a method for manufacturing particles of active ingredients, meeting all the above requirements and further improving the encapsulation of the active ingredients.

This method implements one or many whey protein(s), after they are subjected to a denaturation treatment.

By whey proteins according to the invention it is generally meant whey protein of cow's milk, however, these same proteins derived from milk of another mammal such as goat. The protein of cow's milk are composed of 80% casein, a protein likely to coagulate in acid medium or under the action of the rennet by letting a liquid, the whey which contains other milk proteins, mainly lactalbumin and lactoglobulin. The whey proteins are those selected according to the invention. Indeed, besides their emulsifying and gelling properties such as they have been revealed in the frame of the method of the invention, these proteins have a very high nutritional value for humans or animals; they can also be assimilated and metabolized by the human or animal organism.

The whey proteins are mainly the following: beta-lactoglobulin, alpha-lactalbumin, alpha-S1 and -S2 caseins, beta-, gamma- and kappa- caseins, bovine serum albumin, immunoglobulins IgG, IgA, IgM, IgE and IgD, lactoferrin and proteose peptone.

### BRIEF SUMMARY

Thus, the invention concerns a method for manufacturing particles of a fat-soluble and hydrophobic active ingredient, using whey proteins. It provides first the preparation of a double emulsion of said active ingredient, then the obtaining of particles from this emulsion.

The invention therefore provides a method for preparing a double emulsion of an active ingredient, such as vitamin A, said method comprising the following steps:

(a) Providing an aqueous solution of at least one whey protein;

(b) Providing an aqueous solution of at least one anionic polysaccharide;

(c) Subjecting the whey protein(s) to a denaturation treatment;

(d) Hot-preparing an o/w emulsion of the active ingredient in the solution (c), then hot-preparing an (o/w)/w emulsion of the o/w emulsion in the solution (b), or

(e) Preparing an w/w emulsion of the solutions (c) and (b), then hot-preparing an o/(w/w) emulsion of the active ingredient in the w/w emulsion.

In order to obtain the particles from the emulsion (d) or (e), the method above is completed by a step (f) according to which the emulsion obtained in step (d) or (e) is subjected to an encapsulation step to manufacture the particles of said active ingredient.

As previously mentioned, this method allows meeting all the mentioned requirements, in particular, it allows manufacturing stable particles with high content of said active ingredient and constituting a highly bio-available form of the active ingredient.

### BRIEF DESCRIPTION OF THE DRAWING

The single FIGURE represents a comparison of the release of vitamin A between particles according to the invention and particles according to the prior art, as a function of time and pH.

### DETAILED DESCRIPTION

The invention will now be exposed in more detail, with a more precise description of each of steps (a) to (e) and (f) of the methods of the invention and the presentation of preferential variants.

As previously mentioned, the proteins involved in step (a) of the methods are those of the whey. The whey is a by-product of cheese and casein industry, commonly called “skim milk” and obtained after separation by precipitation of milk caseins. On average, 1 L of whey contains about 65 g of solid compounds, including mainly lactose (70-80%), minerals (9%) and proteins remained in solution in the serum of the milk after the precipitation of caseins (9%). They represent 15 to 20% of total proteins of a bovine milk. The major protein constituents are (β-lactoglobulin ((β-LG), α-lactalbumin (α-LA), immunoglobulins (Ig), bovine serum albumin (BSA), sodium caseinate, and proteose peptones coming from the degradation of (β-casein (10 to 20%). At lower concentrations, (β-casein is also found, as well as other various proteins such as lactoferrin (LF), lactollin and transferrin.

Whey proteins are commercially available. Thus, a BiPRO® Whey Protein Isolate WPI is found, provided by Davisco (USA) and containing 92.4% of highly purified milk proteins, including 72% of (β-LG, 14.4% of α-LA and 4.1% of BSA. Whey Protein Concentrate WPC is also found, less rich in milk proteins than the WPI and containing from 35 to 80% thereof, according to the suppliers. Among these, WPC35 SICAPRO®, provided by Euroserum (France), WPC60-WPC80 MILEI®, provided by Milei (Germany) may be mentioned. The loss of milk proteins, in these concentrates, is compensated by the presence of lactose (between 8 and 40%, against <1% in the BiPRO® WPI), fats and minerals and salts.

According to the invention, the proportion of total proteins in the whey is preferably of at least 30% (w/w, by dry weight), advantageously, it is of at least 60%, even better, of at least 80%, and even of at least 90%. The authors effectively observed that higher this proportion, stronger the protection and bioavailability of the active ingredient in the obtained particles. As previously mentioned, the protein fraction of whey is rich in (β-LG; preferably, according to the invention, this proportion reaches at least 66% (w/w, by dry weight). Nevertheless, a lower proportion in said proteins, for example, of at most 25% (w/w, by dry weight) may advantageously be offset by the presence of lactose which thus allows significantly increasing the nutritional value of the product.

In order to be used in the frame of the method of the invention, whey proteins have to be denatured. The authors observed that this step allows increasing, in an unexpected way, the emulsifying properties of these proteins. They are denatured in conditions known by those skilled in the art. However, the denaturation is preferably conducted by heat treatment, at a temperature from 70° to 80°, for at least 15 minutes. A heating at 80° C. for 30 minutes causes a complete denaturation. According to the invention, by denaturation of whey protein(s), at least 80% of said proteins are denatured. Advantageously, at least 90% are denatured and in an optimal method, the totality of the involved whey proteins is denatured.

According to the method of the invention, an emulsion can be obtained, then by shaping this emulsion, particles of any fat-soluble and hydrophobic active ingredient can be obtained; without being restricted thereto, it is particularly suitable for manufacturing vitamin A or vitamin E particles. Advantageously, the vitamin is diluted in an oil, for example rapeseed oil before being dispersed.

In parallel, according to step (b) of the method, an aqueous solution of at least one anionic polysaccharide is provided. These have to be compatible with the whey proteins, they have to facilitate obtaining a double emulsion according to step (d) or (e), and to be capable of forming a gel in view of step (f) of the particles manufacturing method. To this purpose, they are preferably chosen among pectins, and in particular low methylated pectins, alginates, carrageenans such as kappa-carrageenans, xanthan and Gellan gum, as well as any mixture thereof.

Pectins are polymers of plant origin, mainly composed of a chain of links α-(1-4) of D-galacturonic acid (GA) which may be esterified by methanol, or amidated. The esterification degree, or methylation (—COOCH₃), abbreviated DE, and amidation degree (—CONH₂) abbreviated DA, of the GA is defined as the number of methylated or amidated carboxylic functions, respectively for 100 GA patterns. Low methylated pectins, with DE less than 50%, are preferred according to the invention. Indeed, they allow obtaining particles by cold ionic gelling, and give the advantage of not affecting milk proteins solubility at a pH from 4 to 6. Such pectins are commercially available; those provided by Cargill, extracted from lemon juice, can be cited, namely:
Unipectin OF 300C® (DE=30: t: 3%; pHᵢₙᵢₜᵢₐₗ=2.7 in aqueous solution) or LMP1,
Unipectin OF 305C® (DE=25%+DA=21%, pHᵢₙᵢₜᵢₐₗ=4.6 in aqueous solution) or LMPA,
Unipectin OF 100C® (DE=3-12%; pHᵢₙᵢₜᵢₐₗ4.7 in aqueous solution) or LMP2.

Advantageously, the weight ratio (by dry weight) of the protein(s) to the anionic polysaccharide(s) varies from 1:2 to 7:1.

Advantageously, the weight ratio (by dry weight) of the active ingredient to the mixture of protein(s) and anionic polysaccharide(s) varies from 0.3:0.7 to 0.6:0.4.

According to step (d) of the method, said active ingredient is mixed with the aqueous protein solution of step (a) and an o/w emulsion of said active ingredient is carried out hot, at a sufficiently low temperature for not degrading the active ingredient but high enough to obtain the emulsion. It varies based on the active ingredient, it is generally comprised between 40 and 60° C. An efficient dispersion is obtained under stirring. The o/w emulsion resulting therefrom is then mixed with the solution (b) by maintaining the temperature between 40 and 60 ° C. and under stirring. The conditions for obtaining an (o/w)/w emulsion may be easily determined by those skilled in the art based on their general knowledge. Specific examples will be described later.

Alternatively in step (d), a w/w emulsion may be carried out according to step (e): solutions of steps (c) and (b) are mixed to obtain under stirring a w/w emulsion. The active ingredient is then added and, in appropriate conditions, an (w/w)/o emulsion is obtained. Advantageous conditions exist in dispersing, at a temperature varying from 40 to 60° C., the active ingredient in the w/w emulsion, and in applying a high shear stirring, to this dispersion. As for the previous step, the conditions for obtaining this (w/w)/o emulsion, may be easily defined by those skilled in the art based on their general knowledge. Specific examples will be described later.

For an optimal use of both milk proteins and anionic polymers, it is preferable to provide for a prior step to the method, according to which the milk protein(s) and the anionic polysaccharide(s) are rehydrated, respectively. This step may be carried out under gentle stirring, for at least one, even few hours, so as not to break the protein aggregates of the former, neither the polymer network of the latter.

In order to obtain according to step (f) particles of the active ingredient from the emulsion obtained in step (d) or (e) above, any classical technique well known and mastered by those skilled in the art may be implemented. It will be in particular selected based on the size of desired particles. Preferably, the manufacturing of particles is carried out by cold ionic gelling, by extrusion of said emulsion, then immersion of the obtained drops in an aqueous solution of ions. Monovalent or divalent ions are preferred, and in particular sodium, potassium, calcium and/or zinc ions. Advantageously, the solution contains zinc acetate. This method allows obtaining particles with a size varying from about 1 to about 2.5 mm.

Step (f) may also be performed by atomization/drying of the emulsion derived from step (d) or (e). This technique allows obtaining particles with smaller sizes in the order of 0.05 to 1 mm.

The resulting particles then undergo the routine separation, purification, solidification, drying, dehydration, etc. treatments.

The invention also concerns particles of a hydrophobic and fat-soluble active ingredient, having a size preferably less than 2.5 mm, comprising at least one whey protein and one anionic polysaccharide. Preferably, the protein(s) constituting these particles is/are protein(s) as described above according to advantageous variants of the method of the invention. Similarly, the anionic polysaccharide(s) and the active ingredient are those defined previously, in the advantageous proportions also indicated. The particles of the invention are advantageously obtained by a method as described above. They present have a spherical and regular shape, with a non-greasy surface, they are not sticky and not agglomerated and possess a low residual moisture.

The characteristics and advantages of the invention will appear from the examples hereinafter illustrating the particles of the invention and their obtaining method, in support of the annexed figure which represents a comparison of the release of vitamin A between particles according to the invention and particles according to the prior art, as a function of time and pH.

### EXAMPLE 1

### Manufacturing of Vitamin A Particles According to the Invention According to Steps a), b), c), d) and f)

The used ingredients and their proportions (in w/w) in the obtained particles are the following:

| | |
|---|---|
| BiPRO ® WPI | 28.1% |
| LMPI Pectin | 9.3% |
| Vitamin Charge | 58.9% |
| (Vitamin A + Butylhydroxytoluene, BHT) | |
| Including Vitamin A | 47.2% |
| Water | 3.6% |

The WPI is put into solution in water to obtain a concentration of 12% (w/v), and is dispersed under gentle stringing (300 revolutions/min) for at least 2 hours. Separately, the pectin is subjected to the same rehydration treatment, the concentration of the solution being of 4% (w/v). The solutions are then allowed to rest overnight at room temperature. Each solution is briefly stirred, before use.

The WPI solution is subjected to a heat treatment, by heating in a water bath for 30 minutes at 80° C., for denaturation. The pH of the solution is in the order of 7.

In this protein solution, vitamin A is dispersed, then hot-emulsified at 50° C., for 10 minutes using an Ultra-Turrax® T25 (IKA) high shear stirrer at 6800 revolutions/min based on the apparent viscosity (η) of the emulsion for a fine and efficient dispersion of blood cells. Maintaining the emulsion hot, using a water bath, allows reducing the viscosity of the emulsion and overcoming the premature gelling of the emulsion.

This simple oil-in-water emulsion (o/w) is afterwards diluted by adding an amount equal to the protein, an aqueous solution of pectin (1: 1, v/v) for 5 minutes under moderate stirring (760 revolutions/min, always at 50° C. The weight ratio of the protein to the pectin is of 2.8:1.

The loading rate in vitamin A may vary from 10 to 20% of the weight of the final emulsion.

The diluted emulsion remains under gentle stirring (300 revolutions/min) before being extruded and this until the fall of the last drop.

The diluted emulsion is transferred through a peristaltic pump or a syringe pump, by a pipe until a syringe fitted with a needle or a vibrating nozzle. The emulsion drops fall into a bath of divalent cations (Ca²⁺) at 10% (w/v) under continuous and moderate magnetic stirring.

The gelled beads are then collected by vacuum filtration and washed with distilled water (300 mL in 2 times from Ca²⁺). Finally, the beads are spread/distributed over a sheet of greaseproof paper, disposed on a tray, to be dried between 48 and 72 h (according to the extrusion device) in a ventilated oven at 37±2° C.

The obtained particles can match an organized structure, in separated layers, a pectin layer being adsorbed at the surface of the protein layer, or at a disorganized structure, in mixed layer in which the pectin and the protein are distributed.

The encapsulation yield of vitamin A is of 92%.

The characteristics of the obtained particles are collected hereinafter:

| Size | 2.0-2.5 | mm |
|---|---|---|
| Relative moisture | 3.6% | |
| Title in Vitamin A | 1.173.400 | UI/g |

### EXAMPLE 2

### Manufacturing of Vitamin A Particles According to the Invention According to Steps a), b), c), d), e) and f)

The used ingredients and their proportions (in w/w) in the obtained particles are the following:

| | |
|---|---|
| BiPRO ® WPI | 17.6% |
| LMPI Pectin | 5.8% |
| Vitamin charge (Vitamin A + BHT) | 73.0% |
| Including vitamin A | 58.4% |
| Water | 3.6% |

The WPI and pectin solutions are prepared as detailed in the example 1.

A water-in-water emulsion (w/w) is carried out at room temperature under magnetic stirring. To this purpose, both solutions, at pH 7, are emulsified for 5 minutes at 4440 revolutions/minute, in an Ultra-Turrax® T25 (IKA) high shear stirrer.

Vitamin A is dispersed at 50° C., then emulsified at 50° C., for 10 min in the stirrer above, at 4400 revolutions/min, to obtain an emulsion o/(w/w).

The emulsion o(w/w) is subjected to a cold ionic gelling: to this purpose, an extrusion then immersion of the obtained drops are carried out in a bath of divalent cations at 0° C. The obtained beads are left to harden under magnetic stringing, for 10 minutes, then a separation by vacuum filtration is carried out and the resulting particles are washed with water. They are then dried in a ventilation oven at 37° C.

The encapsulation yield of vitamin A is of 99%.

The characteristics of the obtained particles are collected below:

| Size | 1.5-2.0 | mm |
|---|---|---|
| Relative moisture | 3.6% | |
| Title in Vitamin A | 1.548.320 | UI/g |

### EXAMPLE 3

### In Vitro Release of Vitamin A from Particles

200 mg of particles obtained in the example 1 are placed in a dissolution bath of acid medium at pH 1.2 (1 L, stomach medium) for 2 h then transferred for 4 h in a bath of phosphate buffer medium at pH 6.8 (1 L, intestinal medium).

Every 30 min, a sampling of 3 mL is performed in order to measure the absorbance and determine the amount of released vitamin A. A blank measurement is performed before the kinetic start (pH 1.2) and before the transfer of particles at pH 6.8. The absorbance measurement is made with a UV/visible spectrophotometer.

The composition of the media is described below:

Stomach medium:

For a fixed volume of 1 L, the medium at pH 1.2 is composed of:

| | | |
|---|---|---|
| NaCl | 2.9 | g |
| HCl (1N) | 85 | mL |
| Sodium ascorbate | 10 | g |
| Triton X100 | 1 | g |

Intestinal medium:

For a fixed volume of 1 L, the medium at pH 6.8 is composed of:

| | | |
|---|---|---|
| NaH₂PO₄ | 10 | g |
| NaOH (1N) | 30 | mL |
| Sodium ascorbate | 10 | g |
| Triton X100 | 1 | g |

The sodium ascorbate and the triton X100 play respectively the roles of antioxidant and surfactant. They are necessary in order to improve the stability of vitamin A and to facilitate its solubilization in polar aqueous medium.

The release results are represented in the figure in comparison with vitamin A particles obtained by a classic method of double emulsion with gelatin.

It is observed that the particles of the invention provide an enhanced protection of vitamin A at a gastric pH and promotes an immediate and less diffuse release of vitamin A at intestinal pH.

1. A method for preparing a double emulsion of a fat-soluble and hydrophobic active ingredient, comprising:
(a) Providing an aqueous solution of at least one whey protein;
(b) Providing an aqueous solution of at least one anionic polysaccharide;
(c) Subjecting the whey protein(s) to a denaturation treatment;
(d) Hot-preparing an o/w emulsion of the active ingredient in the solution, then hot-preparing an (o/w)/w emulsion of the o/w emulsion in the solution, or
(e) Preparing an w/w emulsion of the solutions, then hot-preparing an o(w/w) emulsion of the active ingredient in the w/w emulsion.

2. A method for manufacturing particles of a fat-soluble and hydrophobic active ingredient, comprising:
(a) Providing an aqueous solution of at least one whey protein;
(b) Providing an aqueous solution of at least one anionic polysaccharide;
(c) Subjecting the whey protein(s) to a denaturation treatment;
(d) Hot-preparing an o/w emulsion of the active ingredient in the solution, then hot-preparing an (o/w)/w emulsion of the o/w emulsion in the solution, or
(e) Preparing an w/w emulsion of the solutions, then hot-preparing an o/(w/w) emulsion of the active ingredient in the w/w emulsion; and
(f) Subjecting the emulsion to an encapsulation step to obtain particles of said active ingredient.

3. The method according to claim 1, wherein the whey proteins are chosen among beta-lactoglobulin, alpha-lactalbumin, bovine serum albumin, sodium caseinate, and lactoferrin.

4. The method according to claim 3, wherein the solution is obtained from whey powder in which the proportion of the total proteins is of at least 30% (w/w, by dry weight).

5. The method according to claim 4, wherein the proportion in beta-lactoglobulin in the whey powder is of at least 66% (w/w, by dry weight).

6. The method according to claim 1, wherein the proportion of beta-lactoglobulin in the whey powder is of at most 25% and in that said powder comprises lactose.

7. The method according to claim 1, wherein, in step (c), said protein(s) is/are denatured by a heat treatment at a temperature varying from 70 to 80° C.

8. The method according to claim 1, wherein the active ingredient is chosen among vitamins A and E.

9. The method according to claim 1, wherein the anionic polysaccharide comprises at least one of pectins, alginates, carrageenans including kappa-carrageenan, xanthan and Gellane gum, and any mixture thereof.

10. The method according to claim 9, wherein the anionic polysaccharide comprises a pectin having a methylation degree less than 50%.

11. The method according to claim 1, wherein the weight ratio (by dry weight) of the protein(s) to the anionic polysaccharide(s) varies from 1:2 to 7:1.

12. The method according to claim 1, wherein the weight ratio (by dry weight) of the active ingredient to the mixture of the protein(s) and of the anionic polysaccharide(s) varies from 0.3:0.7 to 0.6:0.4.

13. The method according to claim 1, wherein the protein(s) and the anionic polysaccharide(s) are rehydrated, respectively.

14. The method according to claim 2, wherein, according to step (f), a cold ionic gelling is carried out, by an extrusion of the emulsion obtained in step (d) or in step (e), then immersion of the obtained drops in an aqueous solution of monovalent or multivalent ions, including sodium, potassium, calcium and zinc ions.

15. The method according to claim 2, wherein, according to step (f), an atomization/drying of the emulsion obtained is step (d) or in step (e), is carried out.

16. A particle of a fat-soluble and hydrophobic active ingredient, having a size at most equal to 2.5 mm, comprising at least one whey protein and one anionic polysaccharide.

17. The particle according to claim 16, wherein the protein(s) and its/their proportion(s) are as defined by a method for preparing a double emulsion of a fat-soluble and hydrophobic active ingredient, comprising: (a) Providing an aqueous solution of at least one whey protein; (b) Providing an aqueous solution of at least one anionic polysaccharide; (c) Subjecting the whey protein(s) to a denaturation treatment; (d) Hot-preparing an o/w emulsion of the active ingredient in the solution, then hot-preparing an (o/w)/w emulsion of the o/w emulsion in the solution, or (e) Prearing an w/w emulsion of the solutions then hot-preparing an o(w/w) emulsion of the active in redient in the w/w emulsion; wherein the whey proteins are chosen among beta-lactoglobulin, alpha-lactalbumin, bovine serum albumin, sodium caseinate, and lactoferrin.

18. The particle according to claim 16, wherein the anionic polysaccharide(s) and its/their proportion(s) are as defined by a method for preparing a double emulsion of a fat-soluble and hydrophobic active ingredient, comprising: (a) Providing an aqueous solution of at least one whey protein; (b) Providing an aqueous solution of at least one anionic polysaccharide; (c) Subjecting the whey protein(s) to a denaturation treatment; (d) Hot-preparing an o/w emulsion of the active ingredient in the solution, then hot-preparing an (o/w)/w emulsion of the o/w emulsion in the solution, or (e) Preparing an w/w emulsion of the solutions, then hot-preparing an o(w/w) emulsion of the active ingredient in the w/w emulsion; wherein the anionic polysaccharide comprises at least one of pectins, alginates, carrageenans including kappa-carrageenan, xanthan and Gellane gum, and any mixture thereof.

19. The particle according to claim 16, wherein the active ingredient and its proportion are as defined by a method for preparing a double emulsion of a fat-soluble and hydrophobic active ingredient, comprising: (a) Providing an aqueous solution of at least one whey protein; (b) Providing an aqueous solution of at least one anionic polysaccharide; (c) Subjecting the whey protein(s) to a denaturation treatment; (d) Hot-preparing an o/w emulsion of the active ingredient in the solution, then hot-preparing an (o/w)/w emulsion of the o/w emulsion in the solution, or (e) Preparing an w/w emulsion of the solutions, then hot-preparing an o(w/w) emulsion of the active ingredient in the w/w emulsion; wherein the active ingredient is chosen among vitamins A and E.

20. The particle according to claim 16, obtained by a method for preparing a double emulsion of a fat-soluble and hydrophobic active ingredient, comprising: (a) Providing an aqueous solution of at least one whey protein; (b) Providing an aqueous solution of at least one anionic polysaccharide; (c) Subjecting the whey protein(s) to a denaturation treatment; (d) Hot-preparing an o/w emulsion of the active ingredient in the solution, then hot-preparing an (o/w)/w emulsion of the o/w emulsion in the solution, or (e) Preparing an w/w emulsion of the solutions, then hot-preparing an o(w/w) emulsion of the active ingredient in the w/w emulsion.
